(19) United States
(12) Patent Application Publication
(10) Pub. No.: US 2022/0392621 A1
(43) Pub. Date: Dec. 8, 2022

(54) ARTIFICIALLY INTELLIGENT SMART HOME ASSISTANT FOR HOME-BASED MEDICAL THERAPY

(71) Applicant: Medtronic, Inc., Minneapolis, MN (US)

(72) Inventors: Daniel George, Minneapolis, MN (US); Daniel Bloomberg, Minneapolis, MN (US)

(21) Appl. No.: 17/342,282

(22) Filed: Jun. 8, 2021

(51) Int. Cl. G16H 40/20; G16H 10/60; G16H 40/67; G16H 40/40; G06Q 10/10; G16H 20/60; G16H 20/40; G06N 5/04; G05B 13/02; A61M 1/34

(52) U.S. Cl. CPC G16H 40/20; G16H 10/60; G16H 40/67; G16H 40/40; G06Q 10/1093; G16H 20/60; G16H 20/40; G06N 5/043; G05B 13/0265; A61M 1/3413; A61M 1/3403; G16H 50/20

(57) ABSTRACT

In some examples, a smart assistant device may receive information associated with a previous medical treatment provided to a patient by one or more medical devices. The smart assistant device may determine, based at least in part on the received information and a medical treatment plan for the patient, an adjustment to one or more parameters of an upcoming scheduled medical treatment provided by the one or more medical devices. The smart assistant device may program or otherwise control the one or more medical devices to make the adjustment to the one or more parameters of the upcoming scheduled medical treatment provided by the one or more medical devices.

### TECHNICAL FIELD

This disclosure relates to medical treatments.

### BACKGROUND

Patients with renal disease may receive hemodialysis treatment, which may involve using a medical machine, such as a dialyzer or an artificial kidney, to remove blood from the body, filter waste and extra fluid from the blood, balance electrolytes, and return the blood to the body. Increasingly, medical devices allow patients to receive such dialysis treatments at home without having to go to a dialysis clinic.

### SUMMARY

In general, aspects of this disclosure are directed to techniques for a smart home assistant to manage medical treatments provided by a medical device to a patient outside of a clinic setting, e.g., in the patient's home. For instance, a patient may use a home-based dialysis machine to receive dialysis treatments at home according to a medical treatment plan specified by a clinician that oversees the dialysis treatments of the patient. The medical treatment plan may specify the parameters of the medical treatments provided by a medical device of the patient, such as specifying the timing of such treatments (e.g., the number of treatments a week) and/or the duration of each of the treatments. In the example of dialysis treatments, the parameters can include, for example, the dialysate prescription, pump voltage thresholds of the dialysis machine, and the like.

In one aspect, a method includes receiving, by processing circuitry of a computing device, information associated with a previous medical treatment provided to a patient by one or more medical devices. The method further includes determining, by the processing circuitry and based at least in part on the information, an adjustment to one or more parameters of an upcoming scheduled medical treatment provided by the one or more medical devices for the patient. The method further includes causing, by the processing circuitry, the one or more medical devices to make the adjustment to the one or more parameters of the upcoming scheduled medical treatment.

In another aspect, a computing device includes a memory. The computing device further includes processing circuitry operably coupled to the memory and configured to: receive information associated with a previous medical treatment provided to a patient by one or more medical devices; determine, based at least in part on the information, an adjustment to one or more parameters of an upcoming scheduled medical treatment provided by the one or more medical devices for the patient; and cause the one or more medical devices to make the adjustment to the one or more parameters of the upcoming scheduled medical treatment.

In another aspect, a non-transitory computer-readable storage medium storing instructions that, when executed, cause processing circuitry of a computing device to: receive information associated with a previous medical treatment provided to a patient by one or more medical devices; determine, based at least in part on the information, an adjustment to one or more parameters of an upcoming scheduled medical treatment provided by the one or more medical devices for the patient; and cause the one or more medical devices to make the adjustment to the one or more parameters of the upcoming scheduled medical treatment.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 illustrates an example medical system that includes an example smart assistant device for home-based medical therapy, in accordance with aspects of the present disclosure.

FIG. 2 is a block diagram illustrating further details of an example smart assistant device that assists in the performance of home-based medical treatments, in accordance with one or more aspects of the present disclosure.

FIG. 3 is a flowchart illustrating an exemplary operation of a smart assistant device in accordance with at least one technique disclosed herein.

Like reference characters denote like elements throughout the description and figures.

### DETAILED DESCRIPTION

In examples described herein, a smart home assistant is configured to manage medical treatments provided by a medical device to a patient outside of a clinic setting, e.g., in the patient's home. In some cases, it may be difficult for a patient to know how to comply with the treatment plan specified by the clinician while using a medical device at home for medical treatments. For example, the patient may forget about an upcoming medical treatment and may therefore miss the upcoming treatment. The patient may also not be able to know whether the parameters of the medical treatments being provided by the medical device to the patient is following the parameters set out in the medical treatment. In addition, the medical device may be a very complex piece or pieces of equipment that the patient may find difficult or impossible to control and customize.

In accordance with aspects of this disclosure, a smart home assistant configured to connect to the medical device that provides medical treatments to the patient is configured to help manage the in-home medical treatments of a patient to ensure that the patient is being treated according to the medical treatment plan specified by a clinician that oversees the medical treatments of the patient. In some examples, the smart home assistant is configured to schedule medical treatments for the patient based on the patient's availability and may remind the patient of upcoming medical treatments. In addition, in some examples, the smart home assistant is configured to determine when one or more parameters of a medical treatment provided to the patient does not match the one or more parameters set out in the medical treatment plan, and may automatically control the medical device to adjust the one or more parameters to follow the one or more parameters set out in the medical treatment plan.

Accordingly, aspects of this disclosure may provide one or more potential advantages. By ensuring that the patient is being medically treated by the medical device according to the medical treatment plan specified by a clinician, the smart assistant device may enable the patient to live a healthier life and may increase the chance that the patient experiences a beneficial medical outcome from the medical treatments. Furthermore, by enabling the smart home assistant to determine when one or more parameters of a medical treatment provided by the medical device does not match the one or more parameters set out in the medical treatment plan and to automatically control the medical device to adjust the one or more parameters to follow the one or more parameters set out in the medical treatment plan, the techniques of this disclosure improve the functioning of the medical device by at least ensuring that the medical device operates according to the parameters set out in the medical treatment plan when providing medical treatments to the patient. This may help reduce the amount of time during which the medical device may not operate according to the parameters set out in the medical treatment plan, and increase the effectiveness of medical treatments provided by the medical device.

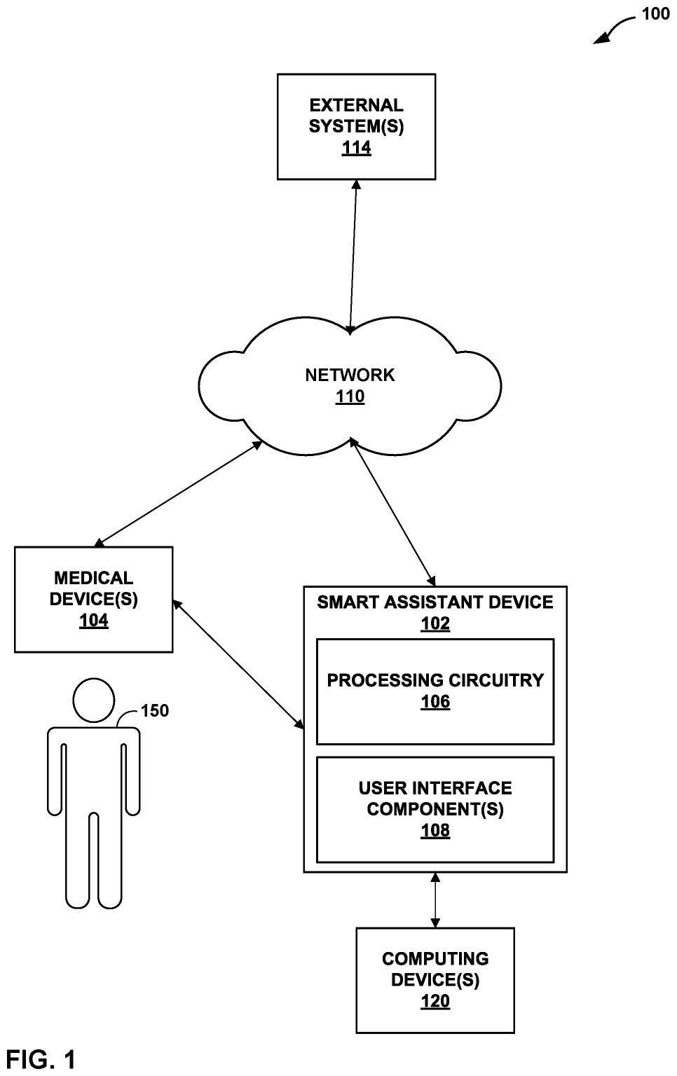

FIG. 1 illustrates an example medical system that includes an example smart assistant device for home-based medical therapy or other medical therapy that takes place outside of a clinic setting and outside of the direct supervision of a clinician. In the example of FIG. 1, system 100 includes smart assistant device 102, one or more medical devices 104, one or more external systems 114, and one or more computing devices 120.

One or more medical devices 104 may be used to treat one or more medical conditions of patient 150. For example, one or more medical devices 104 may be in-home medical devices used in patient 150's home to provide medical treatment to patient 150 without supervision by a clinician during the medical treatment. An example of one or more medical devices 104 may be a dialysis machine for performing in-home dialysis, such as hemodialysis or peritoneal dialysis, an insulin pump, a sleep apnea machine (e.g., a continuous positive airway pressure machine, a pulse oximeter, an external neural stimulator, intravenous equipment, at home physical therapy equipment, and/or rehabilitation equipment.

Smart assistant device 102 is a computing device that is configured to provide smart assistant or virtual assistant functionality to users of smart assistant device 102, such as patient 150 or a caretaker for patient 150. Some references to patient 150 here may also refer to a patient caretaker, such as when referring to receiving input via user interface components 108 and/or generating a notification. Smart assistant device 102 may be placed in patient 150's home to be used by patient 150 in conjunction with one or more medical devices 104. Examples of smart assistant device 102 include a smart speaker, a smart display, and the like. In some examples, smart assistant device 102 may be any suitable computing device that implements smart assistant and/or virtual assistant functionality, such as a smartphone, a laptop computer, a tablet computer, a desktop computer, a television, or the like that performs smart assistant and/or virtual assistant functionality.

Smart assistant device 102 includes processing circuitry 106. Processing circuitry 106, as well as other processors, processing circuitry, controllers, control circuitry, and the like, described herein, may include one or more processors. Processing circuitry 106 may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 106 may include multiple components, such as any combination of one or more microprocessors, one or more DSPs, one or more ASICs, or one or more FPGAs, as well as other discrete or integrated logic circuitry, and/or analog circuitry.

Smart assistant device 102 also includes one or more user interface components 108. One or more user interface components 108 may include a display, a speaker, and the like. For example, the display may be a monitor, cathode ray tube display, a flat panel display such as a liquid crystal (LCD) display, a plasma display, a light emitting diode (LED) display, and/or any other suitable display, and the speaker may be any means for projecting audio to a user. One or more user interface components 108 may also include one or more input devices, such as a keyboard, a mouse, a touch screen, buttons, switches, a microphone, a joystick, a touch pad, or any other suitable input device or combination of input devices.

In some examples, smart assistant device 102 may be configured to perform the functionalities of a smart speaker, such that smart assistant device 102 is configured to receive voice input from a user, such as patient 150, and to perform an action based on the voice input, such as by audibly outputting audio output, such as in the form of speech output, to response to the voice input. For example, one or more microphones in smart assistant device 102 may receive voice input from patient 150 and may, in response, output information to service the voice input, such as by outputting a relevant response to a query contained in the voice input.

Because smart assistant device 102 may receive, store, and/or otherwise determine medical information associated with patient 150, in some examples, smart assistant device 102 may perform voice authentication of voice input received by smart assistant device 102 to determine whether the voice input received by smart assistant device 102 is from an authorized user, such as patient 150. If smart assistant device 102 is unable to authenticate a voice input received by smart assistant device 102 as being from an authorized user, then smart assistant device 102 may refrain from performing actions to service the voice input, such as by refraining from outputting information associated and/or related to medical information associated with patient 150.

Smart assistant device 102 may be configured to perform voice authentication via any suitable technique. For example, smart assistant device 102 may, during a setup process, prompt patient 150 (or other user, such as a patient caretaker) to provide voice input, such as by saying a set of phrases specified by smart assistant device 102. Smart assistant device 102 may extract a set of features from the voice input provided by patient 150 and may store the set of features in memory. When smart assistant device 102 subsequently receives voice input, smart assistant device 102 may extract a set of features from the received voice input and compare the extracted features to the stored features to determine whether the received voice input is from patient 150. For example, if smart assistant device 102 determines that the difference between the extracted features and the stored features are within a specified threshold (e.g., 10%, 15%, etc.), then smart assistant device 102 may successfully authenticate the received voice input as being from patient 150. In other examples, smart assistant device 102 may perform authentication to determine whether a user is an authorized user of smart assistant device 102 via any other suitable authentication techniques, such as facial recognition, fingerprint recognition, and the like.

One or more external systems 114 may include servers, computing devices, and the like, including cloud-based systems, associated with the treatment of patient 150's one or more medical conditions. For example, one or more external systems 114 may include a system used by a clinician that is directing and/or supervising the medical treatments of patient 150 and may store medical information associated with patient 150, such as patient 150's medical records, treatment plans for patient 150, and the like. In another example, one or more external systems 114 may include cloud-based information associated with patient 150, such as calendars associated with patient 150.

Smart assistant device 102 may be configured to communicate with one or more external systems 114 via network 110. Smart assistant device 102 may also connect to one or more medical devices 104 to receive information associated with medical treatments provided by one or more medical devices 104 to patient 150. In some examples, smart assistant device 102 may directly connect to one or more medical devices 104 via a wired or wireless connection, such as via Wi-Fi, Bluetooth, Ethernet, and the like. In some examples smart assistant device 102 may connect to one or more medical devices 104 via network 110. One or more medical devices 104 may also communicate with one or more external systems 114 via network 110.

Network 110 represents any public or private communications network, for instance, cellular, and/or other types of networks, for transmitting data between computing systems, servers, and computing devices. Network 110 may include one or more network hubs, network switches, network routers, or any other network equipment, that are operatively inter-coupled thereby providing for the exchange of information between smart assistant device 102, one or more external systems 114, and/or one or more medical devices 104 via, e.g., the Internet. Smart assistant device 102, one or more external systems 114, and/or one or more medical devices 104 may transmit and receive data across network 110 using any suitable communication techniques. Smart assistant device 102, one or more external systems 114, and/or one or more medical devices 104 may each be operatively coupled to network 110 using respective network links. The links coupling smart assistant device 102, one or more external systems 114, and/or one or more medical devices 104 to network 110 may be Ethernet or other types of network connections and such connections may be wireless and/or wired connections.

Smart assistant device 102 may connect to and communicate with one or more computing devices 120 via wired or wireless connections. Computing devices 120 may include personal computing devices of patient 150 or a patient caretaker, such as a smart phone, a wearable device such as a fitness tracker and/or a smart watch, televisions, laptop computers, desktop computers, tablet computers, and the like.

In accordance with aspects of the present disclosure, smart assistant device 102 may be configured to monitor the medical treatments of patient 150 provided by one or more medical devices 104 and to proactively and automatically perform actions associated with the medical treatments of patient 150 to improve the therapeutic outcomes for patient 150. In some examples, smart assistant device 102 may notify patient 150 and/or a clinician with information regarding the medical treatments for patient 150, adjust the parameters of the medical treatments provided by one or more medical devices 104, and the like.

Many variables may impact the therapeutic efficacy of dialysis as well as many other medical treatments. For example, in the case of dialysis, the duration of a particular dialysis session, the frequency of dialysis sessions, the prescription applied by a dialysis machine during a dialysis treatment, the blood flow rate into a dialysis machine, a dialysate flow rate applied by a dialysis machine, an ultrafiltration rate and volume, heparin rate and volume, blood pressure, patient body temperature and dialysis fluid temperature, dialysate pressure, pump voltage thresholds, and the like, may all impact the efficacy of a particular dialysis treatment. Smart home device 102 considers one or more of these variables and, based on these variables, determines adjustment to one or more parameters of medical treatment provided by one or more medical devices 104 in a more real-time manner and without requiring patient 150 to go to a clinic to meet with a clinician in person.

Processing circuitry 106 of smart assistant device 102 may be configured to determine medical information associated with patient 150, such as the medical history of patient 150, the medical treatment plans for patient 150, information regarding previous medical treatments, information regarding upcoming medical treatments for patient 150 (e.g., a schedule of upcoming medical treatments), and the like. Smart assistant device 102 may receive such medical information associated with patient 150 from one or more external systems 114 via network 110, via user input at one or more user interface components 108, and/or from one or more computing devices 120, and may store such information in memory.

Processing circuitry 106 of smart assistant device 102 may also be configured to determine any other relevant information associated with patient 150 and may store such information in memory of device 102 or otherwise connected to device 102. For example, processing circuitry 106 of smart assistant device 102 may be configured to determine biometric information, such as heart rate, activity level, sleep schedule, blood oxygen level, blood sugar level, and the like regarding patient 150, such as by receiving such biometric information from or more sensors configured to sense these parameters, which may be part of a wearable device of one or more computing devices 120, or separate sensors that are implanted or external to patient 150.

When patient 150 undergoes medical treatment at one or more medical devices 104, such as when patient 150 is receiving a scheduled dialysis treatment, one or more medical devices 104 that are providing the medical treatment to patient 150 may communicate with smart assistant device 102, either via a direct connection or via network 110, to transmit information associated with the medical treatment being provided by one or more medical devices 104 to smart assistant device 102. In some examples, one or more medical devices 104 that are providing the medical treatment to patient 150 may also communicate with one or more external systems 114 via network 110 to transmit such information to one or more external systems 114. In examples in which one or more medical devices 104 include a dialysis machine configured to provide a dialysis treatment to patient 150, one or more medical devices 104 may transmit information regarding the parameters of the dialysis treatment, such the treatment duration, blood flow rate, dialysate flow rate and prescription, ultrafiltration rate and volume, heparin rate and volume, blood pressure of patient 150, the temperature of patient 150, the oxygen saturation level of patient 150, the amount of urea removed from patient 150, the dialyzer clearance, the dialysate pressure, pump voltage thresholds of the dialysis machine, or information regarding any other suitable parameters of patient 150 or combination of the aforementioned parameters.

Smart assistant device 102 may receive from one or more medical devices 104 such information associated with the medical treatment provided by one or more medical devices 104. In some examples, if one or more medical devices 104 do not also transmit information associated with the medical treatment provided by one or more medical devices 104 to one or more external systems 114, then smart assistant device 102 may transmit the information associated with the medical treatment provided by one or more medical devices 104 received by smart assistant device 102 to one or more external systems 114 via network 110.

In some examples, as patient 150 undergoes medical treatment provided by one or more medical devices 104, one or more medical devices 104 may communicate with smart assistant device 102 to send, to smart assistant device 102, information associated with the medical treatment in real-time. Smart assistant device 102 may receive such real-time medical treatment information provided by one or more medical devices 104 as information associated with the medical treatment provided by one or more medical devices 104, and may output the real-time information associated with the medical treatment provided by one or more medical devices 104.

In some examples, smart assistant device 102 may output, for display at a display of smart assistant device 102, a graphical user interface (GUI) that includes graphical representations of real-time information associated with the medical treatment provided by one or more medical devices 104 as one or more medical devices 104 provides the medical treatment to patient 150, such as real-time values of parameters such as the current treatment duration, blood flow rate, dialysate flow rate and prescription, ultrafiltration rate and volume, heparin rate and volume, blood pressure of patient 150, the temperature of patient 150, the oxygen saturation level of patient 150, the amount of urea removed from patient 150 so far, the dialyzer clearance, the dialysate pressure, pump voltage thresholds of the dialysis machine, an amount of time remaining in the treatment session, and the like. In some examples, smart assistant device 102 may send the real-time information associated with the medical treatment provided by one or more medical devices 104 to one or more computing devices 120, and one or more computing devices 120 may output, for display, a GUI that includes graphical representations of real-time information associated with the medical treatment provided by one or more medical devices 104.

As smart assistant device 102 receives real-time information associated with the medical treatment provided by one or more medical devices 104 as one or more medical devices 104 provides the medical treatment to patient 150, smart assistant device 102 may determine, based on the real-time information, whether to output one or more notifications to patient 150. Examples of such real-time notifications may include notifications regarding upcoming medical treatments, which may be outputted within a certain duration prior to the start of the upcoming medical treatments, notifications about the end of the medical treatment, errors or warnings that might occur during the medical treatment, such as dialysate composition issues, bubble detection in blood, that replenishment of resources is required, and the like for a dialysis treatment, notifications about missed treatments and the need to adjust for schedule to account for missed treatments, notification in advance to adjust medication/prescriptions, and/or notifications for patient 150 to make a clinical visit for in-clinic consultation, such as due to indicators (e.g., high blood pressure, low blood sugar, etc.) identified by one or more medical devices 104 and/or smart assistant device 102. In some examples, the notifications may also include guidance for patient 150 regarding how to correct error states and/or alarms that are triggered on one or more medical device 104.

Smart assistant device 102 can present the notifications using any suitable output device. In some cases, smart assistant device 102 selects an output device from a plurality of output devices based on the known location or activity of patient 150. For example, if smart assistant device 102 determines that patient 150 is using a particular device (e.g., a television), then smart assistant device 102 may present the output via the specific device. This may help increase the possibility that patient 150 receives the notification.

As patient 150 undergoes medical treatment provided by one or more medical devices 104, processing circuitry 106 of smart assistant device 102 may be configured to also receive indications of input from patient 150, such as at a keyboard, touchscreen, a microphone, and the like of one or more user interface components 108 of smart assistant device 102. The input from patient 150 can be feedback related to the medical treatment, such as that input that indicates the therapeutic efficacy of the medical treatment, and/or input that smart home assistant device 102 may use to adjust one or more parameters of treatment provided by one or more medical device(s). For example, smart assistant device 102 may receive input from patient 150 that indicates how patient 150 is feeling during the medical treatment, and smart assistant device 102 may send an indication of the voice input to one or more external systems 114 for review by a clinician. If smart assistant device 102 receives voice input from patient 150, processing circuitry 106 may perform speech-to-text functionality to transcribe the voice input received from patient 150 and may send the transcription of the voice input to one or more external systems 114 for review by the clinician.

Processing circuitry 106 of smart assistant device 102 may be configured to determine, based at least in part on the information associated with the medical treatment provided by one or more medical devices 104 and a medical treatment plan for patient 150, an adjustment to the one or more parameters of the next scheduled medical treatment of patient 150. Such information may include the treatment time, the dialysate flow rate, the blood flow rate, the ultrafiltration rate, the ultrafiltration volume, the blood sodium concentration, and the blood hematocrit levels. Processing circuitry 106 may apply one or more heuristics, such as by using a machine-trained neural network, a decision tree, or a set of logic rules stored in memory of smart assistant device 102, to determine, based on such information or any other relevant information, an adjustment to the one or more parameters of the next scheduled medical treatment of patient 150. For example, processing circuitry 106 may adjust one or more of: the treatment time, the dialysate flow rate, the blood flow rate, the ultrafiltration rate, the ultrafiltration volume, the blood sodium concentration, and the blood hematocrit levels.

If processing circuitry 106 determines that an adjustment should be made to one or more parameters of the next scheduled medical treatment session of patient 150, then processing circuitry 106 may be configured to cause or otherwise enable one or more medical devices 104 to adjust the one or more parameters of the next scheduled medical treatment session of patient 150. In some examples, processing circuitry 106 may cause smart assistant device 102 or computing devices 120 to send a signal to one or more medical devices 104 that causes one or more medical devices 104 to automatically adjust the one or more parameters, e.g., without clinician intervention. In some cases, a memory of system 100 may store predetermined permissible changes, such as a predetermined range of values for one or more therapy parameters (e.g., ultrafiltration volumes or rates, treatment times, dialysate flow rates, blood flow rates into a dialysis machine, or the like) within which the parameters may be adjusted without specific clinician input or approval. Processing circuitry 106 may then cause one or more medical devices 104 to automatically adjust the one or more parameters within the predetermined range of values. In some cases, smart assistant device 102 is configured such that any adjustments to the one or more parameters outside the predetermined range of values require clinician approval of the specific adjustment to implement.

In some examples, processing circuitry 106 may send, to the one or more medical devices 104 via a direct connection between smart assistant device 102 and one or more medical devices 104 or via network 110, an indication of the adjustment should be made to one or more parameters of the next scheduled medical treatment session of patient 150, such as to program one or more medical devices 104 to make the adjustment to one or more parameters of the next scheduled medical treatment session of patient 150. In some examples, one or more medical devices 104 may provide an application programming interface (API) for configuring parameters of one or more medical devices 104, and processing circuitry 106 may use the API to make the determined adjustment to the one or more parameters by invoking one or more functions in the API provided by one or more medical devices 104.

In other examples, rather than causing one or more medical devices 104 to automatically adjust the one or more parameters of the next scheduled medical treatment session, in some examples, processing circuitry 106 generates a notification that causes a user to manually make the adjustment. For example, processing circuitry 106 may output, at a display or a speaker of smart assistant device 102, an indication of an adjustment to one or more parameters of the next scheduled medical treatment session for patient 150 to enable patient 150, patient caretaker, or a clinician to make the adjustment to the one or more parameters of the next scheduled medical treatment session of patient 150. Patient 150 or a patient caretaker may then manually configure one or more medical devices 104 to make the adjustment to the one or more parameters to implement for the next scheduled medical treatment session. In some examples, processing circuitry 106 may be configured to output a notification indicating the adjustment to be made to the one or more parameters of the next scheduled medical treatment session by sending an indication of the adjustment to be made to the one or more parameters of the next scheduled medical treatment session to one or more computing devices 120 for output by one or more computing devices 120.

In some examples, processing circuitry 106 may be configured to determine an adjustment to the treatment duration of the next scheduled medical treatment based at least in part on the medical treatment plan for patient 150 and the information associated with the medical treatment provided by one or more medical devices 104. Specifically, if the information associated with a previous medical treatment provided by one or more medical devices 104 indicates that the values of one or more parameters of the previous medical treatment differ from the corresponding values of the corresponding one or more parameters specified by the medical treatment plan for patient 150, then processing circuitry 106 may adjust the same one or more parameters for the next scheduled medical treatment to make up for the difference between the values of one or more parameters of the previous medical treatment and the corresponding values of the corresponding one or more parameters specified by the medical treatment plan for patient 150.

For example, processing circuitry 106 may determine the duration of each scheduled medical treatment for patient 150 is specified by a treatment plan associated with patient 150. If processing circuitry 106 determines, based on the information associated with the medical treatment provided by one or more medical devices 104, that the most recent scheduled medical treatment for patient 150 had a treatment duration that was less than the duration specified by the treatment plan associated with patient 150, then processing circuitry 106 may increase the duration of the next scheduled medical treatment for patient 150 to make up for the shorted duration of the most recent scheduled medical treatment. In this way, smart assistant device 102 may help patient 150 comply with the treatment plan for patient 150, such as pre-determined dialysis goals, by proactively adjusting one or more parameters of a future medical treatment based on known information about past treatment sessions. In contrast to relying on treatment parameters that remain static throughout all treatment sessions until a next clinic visit, smart home device 102 may enable better personalization of medical treatment provided by one or more medical devices 104 to patient 150 based on the specific situation/facts related to patient 150.

For example, processing circuitry 106 may proactively adjust, for a future medical treatment, one or more of: treatment time, dialysate flow rate, blood flow rate, ultrafiltration volume, ultrafiltration rate, sodium, and bicarbonate prescriptions to meet the pre-determined dialysis goals. Processing circuitry 106 may receive information associated with one or more previous medical treatment provided by one or more medical devices 104 and completed by patient 150, and may determine, based on the information, whether to adjust and/or how much to adjust one or more of those parameters of one or more future medical treatment sessions, to meet one or more pre-determined dialysis goals specified by the treatment plan for patient 150.

For example, if processing circuitry 106 determines that an intended dry weight goal specified in the treatment plan was not met in the scheduled medical treatment, processing circuitry 106 may increase the ultrafiltration rate of a future medical treatment to meet the intended dry weight goal. In some examples, processing circuitry 106 may determine to increase the ultrafiltration rate over the next few medical treatment sessions to meet the dry weight goal, and therefore adjust the parameters of two or more future medical treatment sessions, instead of carrying out the entire adjustment in next following medical treatment session, and in this way optimize the adjustments made to minimize the chance of hypotension for patient 150.

In another example, processing circuitry 106 may be configured to receive physiological data of patient 150 from additional medical devices such as a glucose monitor or cardiac monitor. Processing circuitry 106 may, based on such physiological data of patient 150, adjust the electrolyte concentration or glucose levels in one or more future medical treatment sessions to minimize clinical complications. For example, processing circuitry 106 may select a lower glucose concentration level for the next medical treatment session if the physiological data indicates that patient 150 was bordering hypoglycemia in the last few days (e.g., within the last week).

In some examples, processing circuitry 106 of smart assistant device 102 is also be configured determine an adjustment to one or more parameters of the next scheduled medical treatment based at least in part on patient lifestyle information for patient 150. Such patient lifestyle information may include biometric information for patient 150, dietary information for patient 150, information from patient 150 consulting with a clinician, and the like. The clinician may, based on a visit by patient 150, update the medical treatment plan for patient 150 to adjust one or more parameters to future medical treatments provided by one or more medical devices 104 to patient 150. Processing circuitry 106 may download or otherwise receive such an updated medical treatment plan from one or more external systems 114 and may determine, based on the updated treatment plan, an adjustment to be made to the one or more parameters of the next scheduled medical treatment session. For example, if the dietary information indicates that patient 150's recent diet was composed of potassium rich foods, processing circuitry 106 may, based on such dietary information, adjust (e.g., decrease) the potassium levels dialysate for the next scheduled medical treatment session to meet the require potassium goal for patient 150.

Similarly, one or more external systems 114 may provide a dietary journal that patient 150 may regularly update with details regarding patient 150's diet, including the foods patient 150 ate during meals, the timing of each of patient 150's meals, the snacks consumed by patient 150 between meals and the timing of such snacks, and the like. Processing circuitry 106 may download or otherwise receive such dietary information for patient 150 from one or more external systems 114 and may determine, based on the dietary information for patient 150, an adjustment to be made to the one or more parameters of the next scheduled medical treatment session.

Processing circuitry 106 may analyze the dietary information to determine potassium intake levels, sodium intake levels, calcium intake levels, magnesium intake levels, and/or glucose intake levels of patient 150's recent diet and may therefore adjust the potassium levels, sodium levels, calcium levels, magnesium levels, and/or glucose levels of the next scheduled medical treatment session to meet one or more goals associated with the potassium levels, sodium levels, calcium levels, magnesium levels, and/or glucose level of patient 150. For example, if the dietary information indicates that patient 150's recent diet was composed of potassium rich foods, processing circuitry 106 may, based on such dietary information, adjust (e.g., decrease) the potassium levels dialysate for the next scheduled medical treatment session to meet the require potassium goal for patient 150. In addition, processing circuitry 106 may determine and output one or more recommendations for patient 150's diet and/or lifestyle, such as to consume less protein or to consume less water, based on the dietary information.

In some examples, one or more computing devices 120 may include wearable devices such as a smartwatch, a fitness tracker, and the like that is worn by patient 150 and that may measure and collect various biometric information for patient 150. Such biometric information may include information regarding the activity level of patient 150, the heart rate of patient 150, and/or the sleep schedule of patient 150. Smart assistant device 102 may communicate with such wearable devices worn by patient 150 to receive such biometric information for patient 150, and may determine, based on the biometric information for patient 150, an adjustment to be made to the one or more parameters of the next scheduled medical treatment session.

In some examples, processing circuitry 106 may be configured to determine, based at least in part on the information associated with the medical treatment provided by one or more medical devices 104, whether to place an order additional supplies to be used for future medical treatments provided to patient 150 by one or more medical devices 104. By receiving information from one or more medical devices 104, processing circuitry 106 may track the medical treatments performed by one or more medical devices 104 to determine when to order consumables required for future treatments, as well as the quantities of the consumables. Example consumables can include, for example, accessories (e.g., dialysis tubing used to connect a catheter to a dialysis machine), pharmaceuticals (e.g., dialysate and/or recharge chemicals), and the like. Processing circuitry 106 may interface with one or more online stores via network 110 to place orders of accessories, drugs, and the like required for future treatments performed by one or more medical devices 104.

In another example, processing circuitry 106 may receive dietary information of patient 150 and, based on patient 106 experiencing hypotension, hypertension, or being diabetic, accordingly adjust the future medical treatment provided by one or more medical devices 104 to patient 150. In another example, if processing circuitry 106 determines a higher than intended ultrafiltration volume or wet volume for patient 150, such as based on data from previous medical treatments of patient 150 or from biometric information of patient 150, processing circuitry 106 may adjust the ultrafiltration treatment goal for the next one or more medical treatments for patient 150 and/or may notify patient 150 to reduce fluid consumption prior to the next medical treatment. In another example, if one or more medical devices 104 is able to track and/or measure patient 150's blood bicarbonate levels, processing circuitry 106 may, if patient 150 is a high alkalotic patient, adjust the bicarbonate prescription for patient 150 and/or notify patient 150 to adjust their diet to reduce their alkalotic state.

In some examples, processing circuitry 106 can manage medical treatments for patient 150 (provided by one or more medical devices 104) based on the patient's schedule, which may lead to better compliance by patient 150. Better compliance to medical treatments according to a specific treatment plan (e.g., a specific schedule of treatment sessions) may result in better therapeutic outcomes for patient 150. Processing circuitry 106 can determine the patient's schedule using any suitable technique. For example, processing circuitry 106 of smart assistant device 102 may be configured to determine patient 150's schedule based at least in part on electronic calendar information for patient 150. Smart assistant device 102 may be configured to receive calendar information and/or daily schedules for patient 150 from patient 150's cloud-based calendaring service that is a part of one or more external systems 114, receive calendar information for patient 150 from patient 150's smart phone, and the like.

In some examples, processing circuitry 106 can manage medical treatments for patient 150 based on the patient's schedule by at least, for example, sending patient 150 reminders of schedule treatment sessions or clinic visits, rescheduling treatment sessions as needed (e.g., in response to determining patient 150 missed a treatment session), extending a duration of a particular treatment session based on a skipped or shortened past treatment session, or any combination thereof.

If patient 150's calendar information includes information regarding upcoming scheduled visits to a clinic, then processing circuitry 106 of smart assistant device 102 may be configured to use patient 150's calendar information to remind patient 150 regarding an upcoming visit to the clinic. Processing circuitry 106 of smart assistant device 102 may be configured to inform patient 150 regarding an upcoming scheduled visit to the clinic by outputting, at a display of smart assistant device 102 or at a speaker of smart assistant device 102, a notification indicative of a reminder of the upcoming visit to the hospital or clinic prior to the upcoming scheduled visit to the clinic.

If patient 150's calendar information includes information regarding upcoming scheduled medical treatments for patient 150 provided by one or more medical devices 104, then processing circuitry 106 of smart assistant device 102 may be configured to use patient 150's calendar information to inform patient 150 regarding upcoming scheduled medical treatments provided by one or more medical devices 104. Processing circuitry 106 of smart assistant device 102 may be configured to inform patient 150 regarding an upcoming scheduled medical treatment provided by one or more medical devices 104 by outputting, at a display of smart assistant device 102 or at a speaker of smart assistant device 102, a notification indicative of a reminder of the upcoming scheduled medical treatment prior to the upcoming scheduled medical treatment. For example, processing circuitry 106 may be configured to inform patient 150 regarding an upcoming scheduled medical treatment an hour, thirty minutes, fifteen minutes, ten minutes, five minutes, and the like before the start time of the upcoming scheduled medical treatment. The notification may indicate the date and time of the upcoming medical treatment and may provide an option for patient 150 to accept or reject the scheduling of the upcoming medical treatment. If smart assistant device 102 receives user input indicative of patient 150 rejecting the scheduling of the upcoming medical treatment, then processing circuitry 106 may facilitate the rescheduling of the medical treatment. For example, processing circuitry 106 may determine an alternative future date and/or time for the upcoming medical treatment or may output an indication of one or more alternative date and/or times for the upcoming medical treatment for selection by patient 150.

In some examples, processing circuitry 106 of smart assistant device 102 may be configured to inform patient 150 regarding an upcoming scheduled medical treatment by outputting, for display at a display or a speaker of smart assistant device 102, a notification indicative of a reminder of the upcoming scheduled medical treatment. In some examples, processing circuitry 106 of smart assistant device 102 may be configured to inform patient 150 regarding an upcoming scheduled medical treatment by outputting, for audio output at a speaker of smart assistant device 102, a reminder of the upcoming scheduled medical treatment. In some examples, processing circuitry 106 of smart assistant device 102 may be configured to inform patient 150 regarding an upcoming scheduled medical treatment by sending an indication of the upcoming scheduled medical treatment to one or more computing devices 120, and one or more computing devices 120 may, in response to receiving the indication of the upcoming scheduled medical treatment, output a reminder of the upcoming scheduled medical treatment.

In some examples, processing circuitry 106 is configured to automatically schedule and reschedule upcoming medical treatments for patient 150 provided by one or more medical devices 104 based at least in part on calendar information for patient 150 and a medical treatment plan for patient 150, such as one specified by a clinician. The medical treatment plan for patient 150 may, for example, specify the number of medical treatment sessions for patient 150 per week, the duration of each medical treatment session for patient 150, and the like.

Processing circuitry 106 of smart assistant device 102 may be configured to schedule, based on patient 150's electronic calendar information and the medical treatment plan for patient 150, medical treatments for patient 150 provided by one or more medical devices 104. Processing circuitry 106 of smart assistant device 102 may be configured to determine, based on patient 150's calendar information, dates and times where patient 150 may be free to undergo a medical treatment provided by one or more medical devices 104. Processing circuitry 106 of smart assistant device 102 may be configured to determine, based on the dates and times where patient 150 may be free to undergo a medical treatment provided by one or more medical devices 104, a schedule of medical treatments for patient 150.

For example, a medical treatment plan for patient 150 may specify that patient 150 is to undergo one hour dialysis sessions three times a week. Processing circuitry 106 of smart assistant device 102 may be configured to determine, based on patient 150's calendar information, dates and times, that patient 150 is busy on Mondays, Tuesdays, Thursdays, and is available to undergo one hour dialysis sessions on Wednesdays, Fridays, Saturdays, and Sundays. Processing circuitry 106 may therefore be configured to therefore schedule patient 150 to undergo dialysis sessions on Wednesdays, Fridays, and Sundays (and provide the requisite reminders in some examples).

In some examples, processing circuitry 106 is configured to schedule an upcoming medical treatment for patient 150 at a particular date and time and having a particular duration by updating patient 150's electronic calendar to include an event associated with the upcoming medical treatment at the particular date and time and having the particular duration. For example, smart assistant device 102 may communicate with a cloud calendaring service via network 110 to update patient 150's calendar to include an event associated with the upcoming medical treatment at the particular date and time and having the particular duration.

Processing circuitry 106 may also notify patient 150 that an upcoming medical treatment has been scheduled for patient 150, such as by outputting, at a display of smart assistant device 102 or at a speaker of smart assistant device 102, a notification indicating that an upcoming medical treatment has been scheduled for patient 150. The notification may indicate the date and time of the scheduled upcoming medical treatment and may provide an option for patient 150 to accept or reject the scheduling of the upcoming medical treatment. If smart assistant device 102 receives user input indicative of patient 150 rejecting the scheduling of the upcoming medical treatment, then processing circuitry 106 may determine an alternative date and/or time for the upcoming medical treatment or may output an indication of one or more alternative date and/or times for the upcoming medical treatment for selection by patient 150.

In some examples, processing circuitry 106 of smart assistant device 102 may be configured to determine that patient 150 has missed a scheduled medical treatment provided by one or more medical devices 104. For example, processing circuitry 106 of smart assistant device 102 may determine that patient 150 has missed a scheduled medical treatment provided by one or more medical devices 104 based on smart assistant device 102 not receiving, from one or more medical devices 104, details of a scheduled medical treatment for patient 150, and the like. Because processing circuitry 106 is able to receive or otherwise determine patient 150's electronic calendar information, processing circuitry 106 is able to determine information regarding patient 150's scheduled medical treatments, such as the day and time of each of patient 150's scheduled medical treatments, from the calendar information for patient 150. In some examples, if processing circuitry 106 determines that it has not received, from one or more medical devices 104, information indicative of a scheduled medical treatment for patient 150 by a specified amount of time after the start of the scheduled medical treatment for patient 150, such as by an hour, 90 minutes, two hours, and the like after the start of the scheduled medical treatment, then processing circuitry 106 may be configured to determine that patient 150 has missed a scheduled medical treatment.

Processing circuitry 106 of smart assistant device 102 may be configured to, in response to determining that patient 150 has missed a scheduled medical treatment, output a notification indicating that patient 150 has missed a scheduled medical treatment. In some examples processing circuitry 106 may be configured to output the notification at an output device of one or more user interface components 108 of smart assistant device 102, such as by audibly outputting, at a speaker of smart assistant device 102, an audio message indicating that patient 150 has missed a scheduled medical treatment, or by outputting, for display at a display of smart assistant device 102, a graphical notification indicating that patient 150 has missed a scheduled medical treatment.

In some examples, processing circuitry 106 may output the notification at one or more computing devices 120. As described above, one or more computing devices 120 may be patient 150's personal computing devices, and may include patient 150's smart phone, patient 150's smart watch, patient 150's television, and the like. Smart assistant device 102 may send to one or more computing devices 120 an indication that patient 150 has missed a scheduled medical treatment, thereby causing one or more computing devices 120 to output a notification indicating that patient 150 has missed a scheduled medical treatment.

Processing circuitry 106 of smart assistant device 102 may be configured to, in response to determining that patient 150 has missed a scheduled medical treatment, notify a clinician responsible for the medical care of patient 150 that patient 150 has missed a scheduled medical treatment. For example, processing circuitry 106 may send, via network 110 to one or more external systems 114 used by a clinician responsible for the medical care of patient 150, an indication that patient 150 has missed a scheduled medical treatment.

In some examples, patient 150 missing a scheduled medical treatment may result in a therapy deficiency to patient 150. For example, missing a scheduled medical treatment may cause cramping, fluid overload, low blood pressure, a stroke, and the like. Thus, processing circuitry 106 may, in response to determining that patient 150 has missed a scheduled medical treatment, determine an adjustment of one or more parameters of the upcoming scheduled medical treatment for patient 150 to reduce the therapy deficiency caused by patient 150 missing the scheduled medical treatment. For example, processing circuitry 106 may determine an adjustment in one or more of: ultrafiltration volume, blood flow rate, treatment duration, and the like to meet pre-determined goals such as blood volume, urea reduction ratio (URR), kT/V to make up for the missed medical treatment.

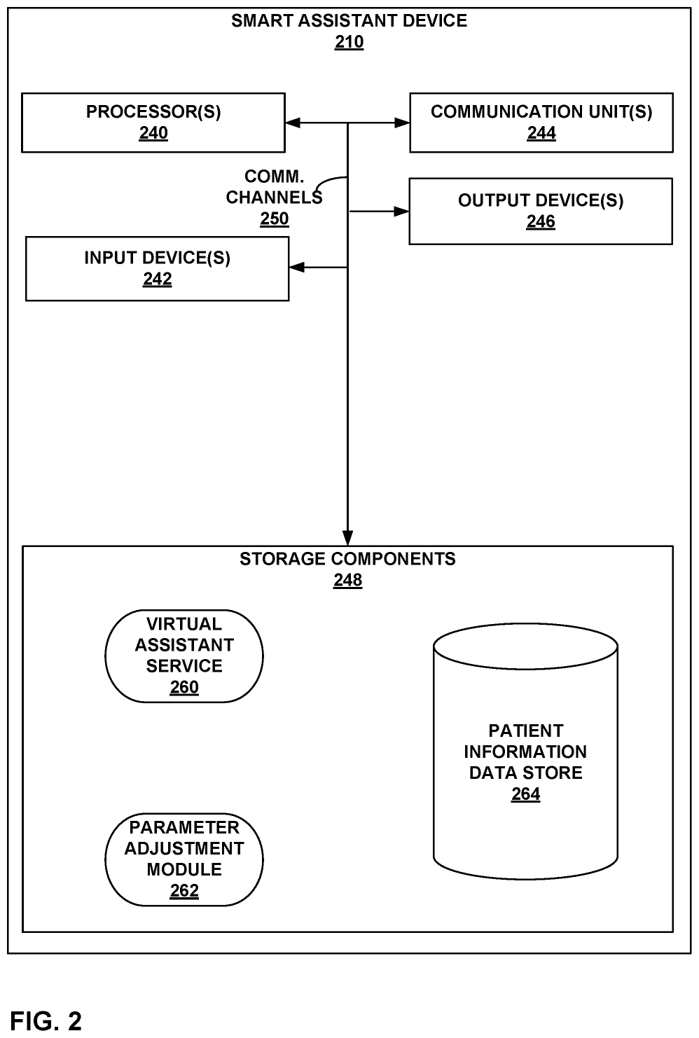

FIG. 2 is a block diagram illustrating further details of an example smart assistant device 202 configured to assist in the performance of home-based medical treatments, in accordance with one or more aspects of the present disclosure. Smart assistant device 202 of FIG. 2 is described below as an example of smart assistant device 102 illustrated in FIG. 1. FIG. 2 illustrates only one particular example of smart assistant device 202, and many other examples of smart assistant device 202 may be used in other instances and may include a subset of the components included in example smart assistant device 202 or may include additional components not shown in FIG. 2.

As shown in the example of FIG. 2, smart assistant device 202 includes one or more processors 240, one or more input devices 242, one or more communication units 244, one or more output devices 246, and storage components 248. Storage components 248 of smart assistant device 202 also include virtual assistant service 260, parameter adjustment module 262, and patient information data store 264.

Communication channels 250 may interconnect each of one or more processors 240, one or more input devices 242, one or more communication units 244, one or more output devices 246, and storage components 248 for inter-component communications (physically, communicatively, and/or operatively). In some examples, communication channels 250 may include a system bus, a network connection, an inter-process communication data structure, or any other method for communicating data.

One or more input devices 242 of smart assistant device 202 are configured to receive user input. Examples of input are tactile, audio, and video input. One or more input devices 242 of smart assistant device 202, in one example, includes a presence-sensitive display, touch-sensitive screen, mouse, keyboard, voice responsive system, video camera, microphone or any other type of device for detecting input from a human or machine.

One or more output devices 246 of smart assistant device 202 are configured to generate an output. Examples of output are tactile, audio, and video output. One or more output devices 246 of smart assistant device 202, in one example, includes a presence-sensitive display, sound card, video graphics adapter card, speaker, cathode ray tube (CRT) monitor, liquid crystal display (LCD), or any other type of device for generating output to a human or machine.

One or more communication units 244 of smart assistant device 202 are configured to communicate with external devices via one or more wired and/or wireless networks by transmitting and/or receiving network signals on the one or more networks. Examples of communication units 244 include a network interface card (e.g. such as an Ethernet card), an optical transceiver, a radio frequency transceiver, a GPS receiver, or any other type of device that can send and/or receive information. Other examples of communication units 244 may include short wave radios, cellular data radios, wireless network radios, as well as universal serial bus (USB) controllers.

One or more processors 240 may implement functionality and/or execute instructions within smart assistant device 202. For example, processors 240 on smart assistant device 202 may receive and execute instructions stored by storage components 248 that execute the functionality of virtual assistant service 260 and parameter adjustment module 262. The instructions executed by processors 240 may cause smart assistant device 202 to store information within storage components 248 during program execution.

Examples of processors 240 include application processors, display controllers, sensor hubs, processing circuitry, and any other hardware configured to function as a processing unit. Processors 240 may execute instructions of virtual assistant service 260 and parameter adjustment module 262 to perform various actions or functions of smart assistant device 202. One or more processors 240 is an example of processing circuitry 106 of FIG. 1.

One or more storage components 248 within smart assistant device 202 may store information for processing during operation of smart assistant device 202 (e.g., smart assistant device 202 may store data, such as patient information data store 264, accessed by virtual assistant service 260 and parameter adjustment module 262 during execution at smart assistant device 202). In some examples, storage component 248 is a temporary memory, meaning that a primary purpose of storage component 248 is not long-term storage. Storage components 248 on smart assistant device 202 may be configured for short-term storage of information as volatile memory and therefore not retain stored contents if powered off. Examples of volatile memories include random access memories (RAM), dynamic random access memories (DRAM), static random access memories (SRAM), and other forms of volatile memories known in the art.

Storage components 248, in some examples, also include one or more computer-readable storage media. Storage components 248 may be configured to store larger amounts of information than volatile memory. Storage components 248 may further be configured for long-term storage of information as non-volatile memory space and retain information after power on/off cycles. Examples of non-volatile memories include magnetic hard discs, optical discs, floppy discs, flash memories, or forms of electrically programmable memories (EPROM) or electrically erasable and programmable (EEPROM) memories. Storage components 248 may store program instructions and/or information (e.g., data) associated with virtual assistant service 260, parameter adjustment module 262 and patient information data store 264.

Patient information data store 264 is a repository configured to store collections of data associated with a patient, such as patient 150 of FIG. 1, that uses smart assistant device 210 to manage and assist the patient's home-based medical therapy. Patient information data store 264 may store the medical treatment plans for the patient, information regarding previous medical treatments performed on the patient, patient lifestyle information for the patient such as biometric information for the patient, dietary information for the patient, information from the patient consulting with a nephrologist, and the like, electronic calendar information associated with patient 150, and the like.

Virtual assistant service 260 is a software application or service that is configured to execute at one or more processors 240 to manage medical treatments provided by a medical device 104 to patient 150 outside a clinic, e.g., in the patient's home. Virtual assistant service 260 may be configured to execute at one or more processors 240 to determine medical information associated with patient 150, such as the medical history of the patient, the medical treatment plans for patient 150, information regarding previous medical treatments, information regarding upcoming medical treatments for patient 150 (e.g., a schedule of upcoming medical treatments), and the like, and to store such information in patient information data store 264. Virtual assistant service 260 may be configured to execute at one or more processors 240 to receive such medical information associated with the patient from one or more external systems, such as one or more external systems 114 of FIG. 1, via one or more communication units 244, via user input at one or more input devices 242, and/or from one or more computing devices used by the patient, such as one or more computing devices 120 used by patient 150 of FIG. 1.

Virtual assistant service 260 may be configured to execute at one or more processors 240 to determine any other relevant information associated with the patient and may store such information in patient information data store 264. For example, virtual assistant service 260 may be configured to execute at one or more processors 240 to determine biometric information, such as heart rate, activity level, sleep schedule, blood oxygen level, blood sugar level, and the like regarding the patient, such as by receiving such biometric information from a wearable device worn by the patient or one or more sensors separate from a wearable device.

When patient 150 undergoes medical treatment delivered by one or more medical devices (e.g., one or more medical devices 104 of FIG. 1), such as when patient 150 is receiving a scheduled dialysis treatment, one or more medical devices that is providing the medical treatment to patient 150 may communicate with smart assistant device 202, either via a direct connection or via a network, to transmit information associated with the medical treatment being provided by the one or more medical devices to smart assistant device 202. In some examples, the one or more medical devices that is providing the medical treatment to patient 150 may also communicate with one or more external systems to transmit such information to the one or more external systems. In examples where the one or more medical devices is a dialysis machine providing a dialysis treatment to patient 150, the one or more medical devices may transmit information regarding the parameters of the dialysis treatment, such the treatment duration, blood flow rate, dialysate flow rate and prescription, ultrafiltration rate and volume, heparin rate and volume, blood pressure of the patient, the temperature of the patient, the oxygen saturation level of the patient, the amount of urea removed from the patient, the dialyzer clearance, the dialysate pressure, pump voltage thresholds of the dialysis machine, and information regarding any other suitable parameters related to the dialysis treatment.

Virtual assistant service 260 may be configured to execute at one or more processors 240 to receive, from the one or more medical devices, such information associated with the medical treatment provided by the one or more medical devices to the patient. In some examples, if the one or more medical devices does not also transmit information associated with the medical treatment provided by the one or more medical devices to one or more external systems used by a clinician responsible for the patient's medical care, then virtual assistant service 260 may be configured to execute at one or more processors 240 to transmit the information associated with the medical treatment provided by the one or more medical devices received by smart assistant device 202 to the one or more external systems via communication units 244.

In some examples, as patient 150 undergoes medical treatment at the one or more medical devices, the one or more medical devices may communicate with smart assistant device 202 to send, to smart assistant device 202, information associated with the medical treatment provided by the one or more medical devices in real-time as the medical treatment occurs. Virtual assistant service 260 may be configured to execute at one or more processors 240 to receive such real-time information associated with the medical treatment provided by the one or more medical devices as information associated with the medical treatment provided by the one or more medical devices, and may output the real-time information associated with the medical treatment provided by the one or more medical devices.

In some examples, virtual assistant service 260 may be configured to execute at one or more processors 240 to output, for display at a display of one or more output devices 246, a graphical user interface (GUI) that includes graphical representations of real-time information associated with the medical treatment provided by the one or more medical devices as the one or more medical devices provides the medical treatment to patient 150, such as real-time values of parameters such as the current treatment duration, blood flow rate, dialysate flow rate and prescription, ultrafiltration rate and volume, heparin rate and volume, blood pressure of patient 150, the temperature of patient 150, the oxygen saturation level of patient 150, the amount of urea removed from patient 150 so far, the dialyzer clearance, the dialysate pressure, pump voltage thresholds of the dialysis machine, and the like. In some examples, virtual assistant service 260 may be configured to execute at one or more processors 240 to send the real-time information associated with the medical treatment provided by the one or more medical devices to one or more computing devices 120 (FIG. 1) used by patient 150 or a patient caretaker, and one or more computing devices 120 may output, for display, a GUI that includes graphical representations of real-time information associated with the medical treatment provided by the one or more medical devices.

As smart assistant device 202 receives real-time information associated with the medical treatment provided by the one or more medical devices as the one or more medical devices provides the medical treatment to the patient, virtual assistant service 260 may be configured to execute at one or more processors 240 to determine, based on the real-time information, whether to output one or more warnings to the patient.

As patient 150 undergoes medical treatment at the one or more medical devices, virtual assistant service 260 may be configured to execute at one or more processors 240 to also receive indications of input from patient 150, such as at a keyboard, touchscreen, a microphone, and the like of one or more input devices 242. For example, smart assistant device 202 may receive, at one or more input devices 242, input from patient 150 related to the medical treatment, such as an indication of how patient 150 is feeling during the medical treatment. Virtual assistant service 260 may be configured to execute at one or more processors 240 to send an indication of the voice input to one or more external systems used by the clinician for review by the clinician. If one or more input devices 242 receives user input in the form of voice input from patient 150, then virtual assistant service 260 may be configured to execute at one or more processors 240 to perform speech-to-text functionality to transcribe the voice input received from patient 150 and may send the transcription of the voice input to the one or more external systems used by the clinician for review by the clinician.

Virtual assistant service 260 may be configured to use parameter adjustment module 262 to determine an adjustment to the one or more parameters of the next scheduled medical treatment of the patient, e.g., as discussed with reference to smart assistance device 102 of FIG. 1. An adjustment to a parameter of a medical treatment of the patient includes changing a value of the parameter from the value previously used in the medical treatment of the patient.

Parameter adjustment module 262 is a software application or service that is configured to execute at one or more processors 240 to determine, based at least in part on the information stored in patient information data store 264 regarding patient 150, such as the information associated with the medical treatment provided by the one or more medical devices and a medical treatment plan for patient 150, an adjustment to the one or more parameters of the next scheduled medical treatment of patient 150. In some examples, parameter adjustment module 262 may be configured to execute or otherwise use one or more heuristics, a machine-trained neural network, a decision tree, a set of logic rules, and the like, to determine, based on the information stored in patient information data store 264 regarding the patient, an adjustment to the one or more parameters of the next scheduled medical treatment of the patient.

If parameter adjustment module 262 determines that an adjustment should be made to one or more parameters of the next scheduled medical treatment session of patient 150, then parameter adjustment module 262 may be configured to execute at one or more processors 240 to enable the one or more medical devices to adjust the one or more parameters of the next scheduled medical treatment session of the patient. In some examples, parameter adjustment module 262 may be configured to execute at one or more processors 240 to output, at a display or a speaker of one or more output devices 246, an indication of an adjustment to one or more parameters of the next scheduled medical treatment session for the patient to enable the patient or a clinician to make the adjustment to the one or more parameters of the next scheduled medical treatment session of patient 150. Patient 150 may therefore configure the one or more medical devices to make the adjustment to the one or more parameters.

In some examples, parameter adjustment module 262 may be configured to execute at one or more processors 240 to send, to the one or more medical devices via a direct connection between smart assistant device 202 and the one or more medical devices or via a network, an indication of the adjustment should be made to one or more parameters of the next scheduled medical treatment session of patient 150, such as to program the one or more medical devices to make the adjustment to one or more parameters of the next scheduled medical treatment session of the patient. In some examples, the one or more medical devices may provide an application programming interface (API) for configuring parameters of the one or more medical devices, and parameter adjustment module 262 may be configured to execute at one or more processors 240 to use the API to make the determined adjustment to the one or more parameters by invoking one or more functions in the API provided by the one or more medical devices.

In other examples, parameter adjustment module 262 may be configured to execute at one or more processors 240 to output, such as for display at a display of one or more output devices 246 for output by a speaker of one or more output devices 246, a notification indicating the adjustment to be made to the one or more parameters of the next scheduled medical treatment session. In some examples, parameter adjustment module 262 may be configured to execute at one or more processors 240 to output a notification indicating the adjustment to be made to the one or more parameters of the next scheduled medical treatment session by sending an indication of the adjustment to be made to the one or more parameters of the next scheduled medical treatment session to one or more computing devices used by the patient.

In some examples, parameter adjustment module 262 may be configured to execute at one or more processors 240 to determine an adjustment to the treatment duration of the next scheduled medical treatment based at least in part on the medical treatment plan for patient 150 and the information associated with the medical treatment provided by the one or more medical devices. For example, parameter adjustment module 262 may be configured to execute at one or more processors 240 to determine the duration of each scheduled medical treatment for patient 150 is specified by a treatment plan associated with the patient. If parameter adjustment module 262 determines, based on the information associated with the medical treatment provided by the one or more medical devices, that the most recent scheduled medical treatment for patient 150 had a treatment duration that was less than the duration specified by the treatment plan associated with the patient, then parameter adjustment module 262 may be configured to execute at one or more processors 240 to increase the duration of the next scheduled medical treatment for patient 150 to make up for the shorted duration of the most recent scheduled medical treatment. Likewise, in some examples, if parameter adjustment module 262 determines, based on the information associated with the medical treatment provided by the one or more medical devices, that the most recent scheduled medical treatment for patient 150 had a treatment duration that was greater than the duration specified by the treatment plan associated with the patient, then parameter adjustment module 262 may be configured to execute at one or more processors 240 to decrease the duration of the next scheduled medical treatment for patient 150 to make up for the increased duration of the most recent scheduled medical treatment.

Parameter adjustment module 262 may also be configured to execute at one or more processors 240 to determine an adjustment to one or more parameters of the next scheduled medical treatment based at least in part on patient lifestyle information for the patient, e.g., as discussed above with respect to smart assistance device 102 of FIG. 1.

Similarly, one or more external systems may provide a dietary journal that the patient may regularly update with details regarding the patient's diet, including the foods the patient ate during meals, the timing of each of the patient's meals, the snacks consumed by the patient between meals and the timing of such snacks, and the like. Virtual assistant service 260 may be configured to execute at one or more processors 240 to download or otherwise receive such dietary information for the patient from one or more external systems, and parameter adjustment module 262 may be configured to execute at one or more processors 240 to determine, based on the dietary information for the patient, an adjustment to be made to the one or more parameters of the next scheduled medical treatment session.

In some examples, the patient may wear one or more wearable devices, such as a smartwatch, a fitness tracker, and the like, that may measure and collect various biometric information for the patient. Such biometric information may include information regarding the activity level of the patient, the heart rate of the patient, the sleep schedule of the patient. Smart assistant device 202 may communicate with such wearable devices worn by the patient to receive such biometric information for the patient, and parameter adjustment module 262 may be configured to execute at one or more processors 240 to determine, based on the biometric information for the patient, an adjustment to be made to the one or more parameters of the next scheduled medical treatment session.

In some examples, as discussed above with smart assistance device 102 of FIG. 1, virtual assistant service 260 may be configured to execute at one or more processors 240 to determine, based at least in part on the information associated with the medical treatment provided by the one or more medical devices, whether to place an order additional supplies to be used for future medical treatments provided to the patient by the one or more medical devices.

In addition, in some examples, as discussed above with smart assistance device 102 of FIG. 1, virtual assistant service 260 may be configured to execute at one or more processors 240 to make adjustments to future treatment sessions based on electronic calendar information for patient 150. Virtual assistant service 260 may be configured to execute at one or more processors 240 to receive calendar information and/or daily schedules for the patient from the patient's cloud-based calendaring service that is a part of one or more external systems 114, receive calendar information for the patient from the patient's smart phone, and the like, and may store such calendar information in patient information data store 264.

If the patient's calendar information includes information regarding upcoming scheduled visits to a clinic or a future treatment session, then virtual assistant service 260 may be configured to execute at one or more processors 240 to use the patient's calendar information to remind the patient regarding an upcoming visit to the clinic or the future treatment session. The reminder can be, for example, output at a display or at a speaker of one or more output devices 246. In addition, as discussed with respect to FIG. 1, in some examples, virtual assistant service 260 may be configured to execute at one or more processors 240 to reschedule or adjust future treatment sessions to account for past missed, shortened, or extended treatment sessions. Virtual assistant service 260 may, for example, be configured to execute at one or more processors 240 to automatically schedule and reschedule upcoming medical treatments for the patient provided by the one or more medical devices based at least in part on calendar information for the patient and a medical treatment plan for the patient. The medical treatment plan for the patient may specify the number of medical treatment sessions for the patient per week, the duration of each medical treatment session for the patient, and the like.

Virtual assistant service 260 may be configured to execute at one or more processors 240 to schedule an upcoming medical treatment for the patient at a particular date and time and having a particular duration by updating the patient's calendar to include an event associated with the upcoming medical treatment at the particular date and time and having the particular duration. For example, virtual assistant service 260 may be configured to execute at one or more processors 240 to communicate with a cloud calendaring service via communication units 244 to update the patient's calendar to include an event associated with the upcoming medical treatment at the particular date and time and having the particular duration. Virtual assistant service 260 may also be configured to execute at one or more processors 240 to notify the patient that an upcoming medical treatment has been scheduled for the patient, such as by outputting, at a display or at a speaker of one or more output devices 246, a notification indicating that an upcoming medical treatment has been scheduled for the patient.

Virtual assistant service 260 may be configured to execute at one or more processors 240 to, in response to determining that patient 150 has missed a scheduled medical treatment, output a notification indicating that patient 150 has missed a scheduled medical treatment. In some examples virtual assistant service 260 may be configured to execute at one or more processors 240 to output the notification at one or more output devices 246, such as by audibly outputting, at a speaker of smart assistant device 202, an audio message indicating that the patient has missed a scheduled medical treatment, or by outputting, for display at a display of smart assistant device 202, a graphical notification indicating that the patient has missed a scheduled medical treatment. Virtual assistant service 260 may also notify a clinician responsible for the medical care of the patient that the patient has missed a scheduled medical treatment.

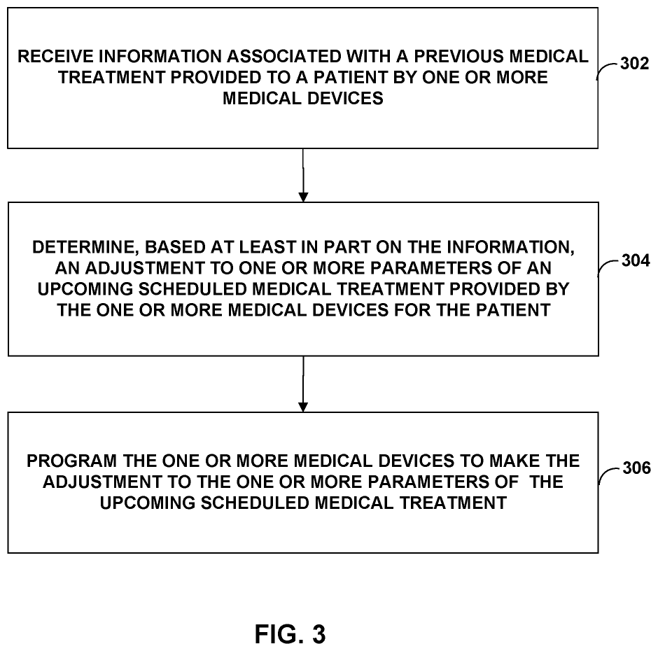

FIG. 3 is a flowchart illustrating an exemplary operation of a smart assistant device in accordance with at least one technique disclosed herein. For purposes of illustration only, the example operations are described below within the context of system 100 and patient 150 of FIG. 1.

As shown in FIG. 3, smart assistant device 102 may receive information associated with a previous medical treatment provided to a patient 150 by one or more medical devices 104 (302). Smart assistant device 102 may determine, based at least in part on the information, an adjustment to one or more parameters of an upcoming scheduled medical treatment provided by the one or more medical devices 104 for the patient 150 (304). Smart assistant device 102 may cause the one or more medical devices 104 to make the adjustment to the one or more parameters of the upcoming scheduled medical treatment (306).

This disclosure includes the following examples:

Example 1: A method includes receiving, by processing circuitry of a computing device, information associated with a previous medical treatment provided to a patient by one or more medical devices; determining, by the processing circuitry and based at least in part on the information, an adjustment to one or more parameters of an upcoming scheduled medical treatment provided by the one or more medical devices for the patient; and causing, by the processing circuitry, the one or more medical devices to make the adjustment to the one or more parameters of the upcoming scheduled medical treatment.

Example 2: The method of example 1, wherein determining the adjustment to the one or more parameters of the upcoming scheduled medical treatment further comprises: determining, by the processing circuitry, that values of the one or more parameters of the previous medical treatment differ from corresponding values of corresponding one or more parameters specified by a medical treatment plan for the patient; and in response to determining that the values of the one or more parameters of the previous medical treatment differ from the corresponding values of the corresponding one or more parameters specified by the medical treatment plan, determining, by the processing circuitry, the adjustment to the one or more parameters of the upcoming scheduled medical treatment.

Example 3: The method of any of examples 1 and 2, further includes determining, by the processing circuitry and based at least in part on the information associated with the previous medical treatment, that the patient has missed the previous medical treatment; and outputting, by the processing circuitry, a notification indicating that the patient has missed the previous medical treatment.

Example 4: The method of example 3, further includes determining, by the processing circuitry, calendar information associated with the patient; and in response to determining that the patient has missed the previous medical treatment, rescheduling, by the processing circuitry and based at least in part on the calendar information associated with the patient and a medical treatment plan for the patient, the previous medical treatment to a future date and time.

Example 5: The method of any of examples 1-4, further includes determining, by the processing circuitry, patient lifestyle information for the patient; and adjusting, by the processing circuitry and based at least in part on the patient lifestyle information for the patient, a schedule of medical treatments provided by the one or more medical devices for the patient.

Example 6: The method of example 5, wherein the patient lifestyle information for the patient comprises biometric information for the patient and dietary information for the patient.

Example 7: The method of any of examples 1-6, further includes determining, by the processing circuitry and based at least in part on the information associated with the previous medical treatment, that the patient has missed the previous medical treatment, wherein missing the previous medical treatment results in a therapy deficiency to the patient, wherein determining the adjustment to one or more parameters of the upcoming scheduled medical treatment comprises adjusting the one or more parameters to reduce the therapy deficiency.

Example 8: The method of example 7, wherein the previous medical treatment comprises a dialysis treatment, and wherein adjusting the one or more parameters to reduce the therapy deficiency comprises at least one of increasing a duration of the upcoming scheduled medical treatment, increasing a dialysate flow rate for the upcoming scheduled medical treatment, increasing a blood flow rate into a dialysis machine for the upcoming scheduled medical treatment, or increasing an ultrafiltration volume for the upcoming scheduled medical treatment.

Example 9: The method of any of examples 1-8, further includes receiving, by the computing device from the one or more medical devices as the one or more medical devices provides a medical treatment for the patient, real-time medical treatment information; and outputting, by the computing device in real-time, the real-time medical treatment information as the one or more medical devices provides the medical treatment for the patient.

Example 10: A computing device includes a memory; and processing circuitry operably coupled to the memory and configured to: receive information associated with a previous medical treatment provided to a patient by one or more medical devices; determine, based at least in part on the information, an adjustment to one or more parameters of an upcoming scheduled medical treatment provided by the one or more medical devices for the patient; and cause the one or more medical devices to make the adjustment to the one or more parameters of the upcoming scheduled medical treatment.

Example 11: The computing device of example 10, wherein to determine the adjustment to the one or more parameters of the upcoming scheduled medical treatment the processing circuitry is further configured to: determine that values of the one or more parameters of the previous medical treatment differ from corresponding values of corresponding one or more parameters specified by a medical treatment plan for the patient; and in response to determining that the values of the one or more parameters of the previous medical treatment differ from the corresponding values of the corresponding one or more parameters specified by the medical treatment plan, determine the adjustment to the one or more parameters of the upcoming scheduled medical treatment.

Example 12: The computing device of any of examples 10 and 11, wherein the processing circuitry is further configured to: determine based at least in part on the information associated with the previous medical treatment, that the patient has missed the previous medical treatment; and output a notification indicating that the patient has missed the previous medical treatment.

Example 13: The computing device of example 12, wherein the processing circuitry is further configured to: determine calendar information associated with the patient; and in response to determining that the patient has missed the previous medical treatment, reschedule, based at least in part on the calendar information associated with the patient and a medical treatment plan for the patient, the previous medical treatment to a future date and time.

Example 14: The computing device of any of examples 10-13, wherein the processing circuitry is further configured to: determine patient lifestyle information for the patient; and adjust, based at least in part on the patient lifestyle information for the patient, a schedule of medical treatments provided by the one or more medical devices for the patient.

Example 15: The computing device of example 14, wherein the patient lifestyle information for the patient comprises biometric information for the patient and dietary information for the patient.

Example 16: The computing device of any of examples 10-15, wherein the processing circuitry is further configured to: determine, based at least in part on the information associated with the previous medical treatment, that the patient has missed the previous medical treatment, wherein missing the previous medical treatment results in a therapy deficiency to the patient, wherein determining the adjustment to one or more parameters of the upcoming scheduled medical treatment comprises adjusting the one or more parameters to reduce the therapy deficiency.

Example 17: The computing device of example 16, wherein the previous medical treatment comprises a dialysis treatment, and wherein to adjust the one or more parameters to reduce the therapy deficiency, the processing circuitry is further configured to at least one of increase a duration of the upcoming scheduled medical treatment, increase a dialysate flow rate for the upcoming scheduled medical treatment, increase a blood flow rate into a dialysis machine for the upcoming scheduled medical treatment, or increase an ultrafiltration volume for the upcoming scheduled medical treatment.

Example 18: The computing device of any of examples 10-17, wherein the processing circuitry is further configured to: receive, from the one or more medical devices as the one or more medical devices provides a medical treatment for the patient, real-time medical treatment information; and output, in real-time, the real-time medical treatment information as the one or more medical devices provides the medical treatment for the patient.

Example 19: A non-transitory computer-readable storage medium storing instructions that, when executed, cause processing circuitry of a computing device to: receive information associated with a previous medical treatment provided to a patient by one or more medical devices; determine, based at least in part on the information, an adjustment to one or more parameters of an upcoming scheduled medical treatment provided by the one or more medical devices for the patient; and cause the one or more medical devices to make the adjustment to the one or more parameters of the upcoming scheduled medical treatment.

Example 20: The non-transitory computer-readable storage medium of example 19, wherein the instructions that cause the processing circuitry to determine the adjustment to the one or more parameters of the upcoming scheduled medical treatment provided by the one or more medical devices for the patient, further cause the processing circuitry to: determine that values of the one or more parameters of the previous medical treatment differ from corresponding values of corresponding one or more parameters specified by a medical treatment plan for the patient; and in response to determining that the values of the one or more parameters of the previous medical treatment differ from the corresponding values of the corresponding one or more parameters specified by the medical treatment plan, determine the adjustment to the one or more parameters of the upcoming scheduled medical treatment.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

Based upon the above discussion and illustrations, it is recognized that various modifications and changes may be made to the disclosed technology in a manner that does not necessarily require strict adherence to the examples and applications illustrated and described herein. Such modifications do not depart from the true spirit and scope of various aspects of the disclosure, including aspects set forth in the claims.

In at least one example, the functions described may be implemented in hardware, software, firmware, or any combination thereof. If implemented in software, the functions may be stored on or transmitted over as at least one instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by at least one computers or at least one processors to retrieve instructions, code and/or data structures for implementation of the techniques described in this disclosure. A computer program product may include a computer-readable medium.

By way of example, and not limitation, such computer-readable data storage media can include RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Combinations of the above should also be included within the scope of computer-readable media.

Instructions may be executed by at least one processors, such as at least one DSPs, general purpose microprocessors, ASICs, FPGAs, CPLDs, or other equivalent integrated or discrete logic circuitry. Accordingly, the term “processor,” as used herein may refer to any of the foregoing structure or any other structure suitable for implementation of the techniques described herein. Also, the techniques could be fully implemented in at least one circuits or logic elements.

Any of the above-mentioned “processors,” “processing circuitry,” and/or devices incorporating any of the above-mentioned processors or processing circuitry, may, in some instances, be referred to herein as, for example, “computers,” “computer devices,” “computing devices,” “hardware computing devices,” “hardware processors,” “processing units,” “processing circuitry,” etc. Computing devices of the above examples may generally (but not necessarily) be controlled and/or coordinated by operating system software, such as Mac OS, iOS, Android, Chrome OS, Windows OS (e.g., Windows XP, Windows Vista, Windows 7, Windows 8, Windows 10, Windows Server, etc.), Windows CE, Unix, Linux, SunOS, Solaris, Blackberry OS, VxWorks, or other suitable operating systems. In some examples, the computing devices may be controlled by a proprietary operating system. Conventional operating systems control and schedule computer processes for execution, perform memory management, provide file system, networking, I/O services, and provide UI functionality, such as GUI functionality, among other things.

The techniques of this disclosure may be implemented in a wide variety of devices or apparatuses, including an integrated circuit (IC) or a set of ICs (e.g., a chip set). Various components, modules, or units are described in this disclosure to emphasize functional aspects of devices configured to perform the disclosed techniques, but do not necessarily require realization by different hardware units.

Various examples have been described. These and other examples are within the scope of the following claims.

What is claimed is:

1. A method comprising:
receiving, by processing circuitry of a computing device, information associated with a previous medical treatment provided to a patient by one or more medical devices;
determining, by the processing circuitry and based at least in part on the information, an adjustment to one or more parameters of an upcoming scheduled medical treatment provided by the one or more medical devices for the patient; and
causing, by the processing circuitry, the one or more medical devices to make the adjustment to the one or more parameters of the upcoming scheduled medical treatment.

2. The method of claim 1, wherein determining the adjustment to the one or more parameters of the upcoming scheduled medical treatment further comprises:
determining, by the processing circuitry, that values of the one or more parameters of the previous medical treatment differ from corresponding values of corresponding one or more parameters specified by a medical treatment plan for the patient; and
in response to determining that the values of the one or more parameters of the previous medical treatment differ from the corresponding values of the corresponding one or more parameters specified by the medical treatment plan, determining, by the processing circuitry, the adjustment to the one or more parameters of the upcoming scheduled medical treatment.

3. The method of claim 1, further comprising:
determining, by the processing circuitry and based at least in part on the information associated with the previous medical treatment, that the patient has missed the previous medical treatment; and
outputting, by the processing circuitry, a notification indicating that the patient has missed the previous medical treatment.

4. The method of claim 3, further comprising:
determining, by the processing circuitry, calendar information associated with the patient; and
in response to determining that the patient has missed the previous medical treatment, rescheduling, by the processing circuitry and based at least in part on the calendar information associated with the patient and a medical treatment plan for the patient, the previous medical treatment to a future date and time.

5. The method of claim 1, further comprising:
determining, by the processing circuitry, patient lifestyle information for the patient; and
adjusting, by the processing circuitry and based at least in part on the patient lifestyle information for the patient, a schedule of medical treatments provided by the one or more medical devices for the patient.

6. The method of claim 5, wherein the patient lifestyle information for the patient comprises biometric information for the patient and dietary information for the patient.

7. The method of claim 1, further comprising:
determining, by the processing circuitry and based at least in part on the information associated with the previous medical treatment, that the patient has missed the previous medical treatment, wherein missing the previous medical treatment results in a therapy deficiency to the patient,
wherein determining the adjustment to one or more parameters of the upcoming scheduled medical treatment comprises adjusting the one or more parameters to reduce the therapy deficiency.

8. The method of claim 7, wherein the previous medical treatment comprises a dialysis treatment, and wherein adjusting the one or more parameters to reduce the therapy deficiency comprises at least one of increasing a duration of the upcoming scheduled medical treatment, increasing a dialysate flow rate for the upcoming scheduled medical treatment, increasing a blood flow rate into a dialysis machine for the upcoming scheduled medical treatment, or increasing an ultrafiltration volume for the upcoming scheduled medical treatment.

9. The method of claim 1, further comprising:
receiving, by the computing device from the one or more medical devices as the one or more medical devices provides a medical treatment for the patient, real-time medical treatment information; and
outputting, by the computing device in real-time, the real-time medical treatment information as the one or more medical devices provides the medical treatment for the patient.

10. A computing device comprising:
a memory; and
processing circuitry operably coupled to the memory and configured to:
receive information associated with a previous medical treatment provided to a patient by one or more medical devices;
determine, based at least in part on the information, an adjustment to one or more parameters of an upcoming scheduled medical treatment provided by the one or more medical devices for the patient; and
cause the one or more medical devices to make the adjustment to the one or more parameters of the upcoming scheduled medical treatment.

11. The computing device of claim 10, wherein to determine the adjustment to the one or more parameters of the upcoming scheduled medical treatment the processing circuitry is further configured to:
determine that values of the one or more parameters of the previous medical treatment differ from corresponding values of corresponding one or more parameters specified by a medical treatment plan for the patient; and
in response to determining that the values of the one or more parameters of the previous medical treatment differ from the corresponding values of the corresponding one or more parameters specified by the medical treatment plan, determine the adjustment to the one or more parameters of the upcoming scheduled medical treatment.

12. The computing device of claim 10, wherein the processing circuitry is further configured to:
determine based at least in part on the information associated with the previous medical treatment, that the patient has missed the previous medical treatment; and
output a notification indicating that the patient has missed the previous medical treatment.

13. The computing device of claim 12, wherein the processing circuitry is further configured to:
determine calendar information associated with the patient; and
in response to determining that the patient has missed the previous medical treatment, reschedule, based at least in part on the calendar information associated with the patient and a medical treatment plan for the patient, the previous medical treatment to a future date and time.

14. The computing device of claim 10, wherein the processing circuitry is further configured to:
determine patient lifestyle information for the patient; and
adjust, based at least in part on the patient lifestyle information for the patient, a schedule of medical treatments provided by the one or more medical devices for the patient.

15. The computing device of claim 14, wherein the patient lifestyle information for the patient comprises biometric information for the patient and dietary information for the patient.

16. The computing device of claim 10, wherein the processing circuitry is further configured to:
determine, based at least in part on the information associated with the previous medical treatment, that the patient has missed the previous medical treatment, wherein missing the previous medical treatment results in a therapy deficiency to the patient,
wherein determining the adjustment to one or more parameters of the upcoming scheduled medical treatment comprises adjusting the one or more parameters to reduce the therapy deficiency.

17. The computing device of claim 16, wherein the previous medical treatment comprises a dialysis treatment, and wherein to adjust the one or more parameters to reduce the therapy deficiency, the processing circuitry is further configured to at least one of increase a duration of the upcoming scheduled medical treatment, increase a dialysate flow rate for the upcoming scheduled medical treatment, increase a blood flow rate into a dialysis machine for the upcoming scheduled medical treatment, or increase an ultrafiltration volume for the upcoming scheduled medical treatment.

18. The computing device of claim 10, wherein the processing circuitry is further configured to:
receive, from the one or more medical devices as the one or more medical devices provides a medical treatment for the patient, real-time medical treatment information; and
output, in real-time, the real-time medical treatment information as the one or more medical devices provides the medical treatment for the patient.

19. A non-transitory computer-readable storage medium storing instructions that, when executed, cause processing circuitry of a computing device to:
receive information associated with a previous medical treatment provided to a patient by one or more medical devices;
determine, based at least in part on the information, an adjustment to one or more parameters of an upcoming scheduled medical treatment provided by the one or more medical devices for the patient; and
cause the one or more medical devices to make the adjustment to the one or more parameters of the upcoming scheduled medical treatment.

20. The non-transitory computer-readable storage medium of claim 19, wherein the instructions that cause the processing circuitry to determine the adjustment to the one or more parameters of the upcoming scheduled medical treatment provided by the one or more medical devices for the patient, further cause the processing circuitry to:
determine that values of the one or more parameters of the previous medical treatment differ from corresponding values of corresponding one or more parameters specified by a medical treatment plan for the patient; and
in response to determining that the values of the one or more parameters of the previous medical treatment differ from the corresponding values of the corresponding one or more parameters specified by the medical treatment plan, determine the adjustment to the one or more parameters of the upcoming scheduled medical treatment.
